(19) United States
(12) Patent Application Publication
(10) Pub. No.: US 2023/0305008 A1
(43) Pub. Date: Sep. 28, 2023

(54) SERS-NANOTAG AND DIAGNOSTIC KIT FOR DETECTING BREAST CANCER BIOMARKERS

(71) Applicant: COUNCIL OF SCIENTIFIC & INDUSTRIAL RESEARCH, New Delhi (IN)

(72) Inventors: Kaustabh Kumar Maiti, Trivandrum (IN); Vishnu Priya Murali, Trivandrum (IN); Varsha Karunakaran, Trivandrum (IN); Deepika Selvakumar, Trivandrum (IN); Madhukrishnan Murali, Trivandrum (IN); Sujathan Kunjuraman, Trivandrum (IN); Saritha Valliamma Neelakantapillai, Trivandrum (IN); Asha Lekshmi, Trivandrum (IN)

(73) Assignee: COUNCIL OF SCIENTIFIC & INDUSTRIAL RESEARCH, New Delhi (IN)

(21) Appl. No.: 18/041,283

(22) PCT Filed: Jun. 14, 2021
(86) PCT No.: PCT/IN2021/050577

§ 371 Date: Feb. 10, 2023

(30) Foreign Application Priority Data
Aug. 11, 2020 (IN) 202011034768

(51) Int. Cl. G01N 33/574; G01N 33/58; G01N 33/532; C07K 16/28; C07K 16/32

(52) U.S. Cl. CPC G01N 33/57415; G01N 33/587; G01N 33/532; G01N 33/57492; C07K 16/2869; C07K 16/32; G01N 2333/723

(57) ABSTRACT

The present invention discloses a SERS-nanotag comprising gold nanoparticle, an encapsulating agent, a Raman reporter and an antibody. The present invention also discloses a diagnostic kit consisting of SERS-nanotags for identification of breast cancer biomarker selected from the group consisting of Estrogen Receptor (ER), Progesterone Receptor (PR), human epidermal growth factor receptor 2 (HER2) and Ki67, simultaneously in abreast cancer tissue sample using a surface enhanced Raman scattering signature peaks. The multiplexing Raman peak pattern provides the presence of multiple biomarkers at a time in heterogeneous paraffin embedded breast cancer tissue samples with a concentration level of the SERS-nanotags by applying single laser (532 nm/633 nm/785 nm) revealing simultaneous Raman peaks for the respective biomarkers.

### FIELD OF THE INVENTION

The present invention relates to a SERS-nanotag comprising gold nanoparticle, an encapsulating agent, a Raman reporter and an antibody. The present invention also relates to a diagnostic kit having the SERS-nanotag for simultaneous detection of multiple breast cancer biomarkers selected from the group consisting of Estrogen Receptor (ER), Progesterone Receptor (PR), Human Epidermal Growth Factor Receptor 2 (HER2) and Ki67 in a paraffin embedded breast tissue sample.

### BACKGROUND OF THE INVENTION

Development of diagnostic SERS-nanoprobes for early and accurate detection of a disease is a challenging task in biomedical research. In the field of bio imaging, diagnostics and drug delivery, many optical imaging technologies are flourished, out of which SERS has emerged as a promising technique for detection of biological and chemical molecules adsorbed on nano roughened metallic surfaces like gold, silver etc. SERS employs the principle of Raman spectroscopy which is based on the inelastic scattering of incident radiation. It allows capturing of unique signatures corresponding to vibrations of molecules and provides signal enhancement up to 10⁸-10¹⁴ folds than the normal Raman spectroscopy which enabled for minute chemical changes in biological samples even in cells and tissues.

Breast cancer is the most common cancer among women. Hormone receptors including Estrogen receptor (ER) and Progesterone receptor (PR) status are key biomolecules in breast cancer. Over-expression of HER2/Neu gene is associated breast cancer patient's prognosis and therapy and Ki67 is a proliferative marker. ER, PR, HER2 and Ki67 panel is essential in an estimation process of breast cancer prognosis which plays a significant role in treatment choice for breast cancer worldwide.

Presence of different biomarkers needs different modes of treatment strategy. Hence, it is very useful to detect the biomarkers quickly in real time and simultaneously. There are few reports on biomarker detection in clinical samples using SERS platform, but no such SERS based biomarker detection kit has been formulated yet especially for HER-2 grading. Salehi et al., in 2014 [Salehi, M., Schneider, L., Strobel, P., Marx, A., Packeisen, J., Schlucker, S. 2014. Two-Color SERS Microscopy for Protein Co-localization in Prostate Tissue with Primary Antibody-Protein A/G-Gold Nanocluster Conjugates. Nanoscale,6(4), pp. 2361-7] reported a formulation in which silica coated gold nanoclusters were used as SERS substrate for the detection of PSA and p63 on non-neoplastic prostrate tissue samples. Whereas, Wang et al., in 2017 [Wang, Y. W., Reder, N. P., Kang, S et al., 2017. Raman-encoded molecular imaging (REMI) with topically applied SERS nanoparticles for intraoperative guidance of lumpectomy. Cancer Research, 77(16), pp. 4506-16] reported a Raman-encoded molecular imaging (REMI) technique where the targeted nanoparticles are topically applied on excised tissues to enable rapid visualization of a panel of cell surface biomarkers at surgical margin on clinical samples. Currently immune-histochemical analysis is followed by pathologists to determine the multiple breast cancer biomarkers. To surmount the disadvantages associated with conventional immunohistochemistry technique such as being highly subjective and time consuming, there is a need for a technique for fast detection of multiple breast cancer biomarkers.

### OBJECTIVES OF THE INVENTION

The main objective of the present invention is to provide a SERS-nanotag comprising colloidal AuNPs, a Raman reporter molecule, a biocompatible polymer and an antibody raised against a biomarker selected from the group consisting of Estrogen Receptor (ER), Progesterone Receptor (PR), Human Epidermal Growth Factor Receptor 2 (HER2) and Ki67 for specific simultaneous detection.

Another objective of the present invention is to provide a diagnostic kit for simultaneous detection of multiple biomarkers selected from the group consisting of Estrogen Receptor (ER), Progesterone Receptor (PR), Human Epidermal Growth Factor Receptor 2 (HER2) and Ki67 in a breast tissue sample by surface enhanced Raman scattering (SERS) modality where each biomarkers is identified by Raman fingerprint of the respective SERS-nanotags of the kit.

Still another objective of the present invention is to provide a tissue processing step and an antigen retrieval step to remove paraffin wax and unmask the antigens from the paraffin embedded breast cancer tissue.

Another objective of the present invention is to provide a SERS analysis i.e. scanning, and imaging to gather information from maximum locations in order to know the abundance of the biomarkers.

Yet another objective of the present invention is to provide a SERS intensity based semi-quantitative system for HER-2 gradation, since an over expression of HER-2 (2+and above from immunohistochemistry grading) is considered by the clinicians to judge the samples as positive.

### SUMMARY OF THE INVENTION

An aspect of the present invention provides a SERS-nanotag comprising:
i. gold nanoparticles having size in the range of 40-50 nm;
ii. an encapsulating agent;
iii. a Raman reporter molecule; and
iv. an antibody

Another aspect of the present invention provides a process for synthesis of the SERS-nanotag comprising the steps of:
a. providing gold nanoparticle having size in the range of 40-50 nm in a solution;
b. concentrating the gold nanoparticles of step (a) by centrifugation at 6000 rpm for 30 minutes followed by addition of 0.05% TWEEN 20 to obtain a stabilized concentrated gold nanoparticle solution;
c. adding a Raman reporter molecule to the concentrated gold nanoparticle solution obtained in step(b) and incubating for 30 minutes followed by addition of an encapsulating agent and incubating for 3 hours to obtain a biocompatible gold nanoparticle solution;
d. concentrating the biocompatible gold nanoparticle solution obtained in step (c) by centrifugation at 10,000 rpm for 10 minutes and removing excess encapsulating agent to obtain a solution;
e. re-suspending the solution obtained in step (d) in a buffer and adding (1-Ethyl-3-(3-dimethylaminopropyl) carbodiimide and sulfo-NHS to obtain a reaction mixture;
f. incubating the reaction mixture obtained in step (e) for 30 minutes, centrifuging and re-suspending in the buffer;
g. adding an antibody to the reaction mixture of step (f) and incubating in a shaker incubator;
h. centrifuging the reaction mixture after incubation and re-suspending in the buffer to obtain the SERS-nanotag.

Yet another aspect of the present invention provides a diagnostic kit for detection of breast cancer biomarker comprising:
I. the SERS-nanotag;
II. xylene;
III. absolute ethanol;
IV. citrate buffer (pH 6.1);
V. phosphate buffer saline;
VI. bovine serum albumin; and
VII. an instructions manual.

Still another aspect of the present invention provides a method for detecting breast cancer biomarker in a tissue sample comprising the steps of:
(i) taking a paraffin embedded formalin fixed tissue sample;
(ii) washing the sample with xylene;
(iii) washing the sample of step (ii) with absolute ethanol followed by washing with 95% ethanol followed by washing with 70% ethanol and then with 50% ethanol to obtain a washed tissue sample;
(iv) treating the washed tissue sample of step (iii) with citrate buffer to obtain a treated tissue sample;
(v) incubating the treated tissue sample of step (iv) with bovine serum albumin and washing with phosphate buffer saline;
(vi) incubating the tissue sample of step (v) with the SERS-nanotag for 30 minutes and washing;
(vii) performing Raman spectroscopy on the tissue sample of step (vi) to take signature peaks; and
(viii) analyzing the peaks to confirm the presence of breast cancer biomarker.

### BRIEF DESCRIPTION OF THE ACCOMPANYING DRAWINGS

The objects and features of the present invention will become apparent from the following description of the invention, when taken in conjunction with the accompanying drawings.

FIG. 1: Flowchart representing the working of screening method

FIG. 2: Synthesis and characterization of AuNPs/AgNPs

FIG. 3: Gold nanoparticle with CV as the Raman reporter: A) Raman spectrum of PEGylated AuNP with CV as the reporter and B) Structure of CV

FIG. 4: Gold nanoparticle with SDL as the Raman reporter: A) Raman spectrum of PEGylated AuNP@SDL and B) structure of SDL

FIG. 5: Gold nanoparticle with MBA as the Raman reporter: A) Raman spectrum of AuNP@PEG@MBA and B) Structure of MBA

FIG. 6: Gold nanoparticle with Py L Et as the Raman reporter: A) Raman spectrum of AuNP@PEG@Py L Et and B) Structure of Py L Et

FIG. 7. SERS single spectral analysis of one biomarker: (A) Bright field image of ER/PR negative HER-2 positive tissue, (B) SERS finger print from the same tissue incubated with AuNP@PEG@CV@Anti-HER-2+AuNP@PEG@SDL@Anti-ER, (C) Immunohistogram of the same tissue stained for HER-2, (D) Bright field image of ER/PR positive HER-2 negative tissue, (E) SERS spectra from the same tissue incubated with AuNP@PEG@CV@Anti-HER-2+AuNP@PEG@SDL@Anti-ER, (F) Immunohistogram of the same tissue stained for ER.

FIG. 8: SERS imaging of breast cancer tissues

FIG. 8a. Simultaneous detection of two biomarkers: SERS images from the breast cancer tissues incubated with AuNP@SDL@PEG@antiER and AuNP@CV@PEG@antiHER2 were taken. (A) Bright field image of ER+/HER2+ tissue, (B) Raman image w.r.t 440 nm, spectral fingerprint, (C) Raman image w.r.t 580 nm spectral fingerprint, (D) Average spectrum from the scan [B, C, D are from the same area of bright field image (A)], (E) Bright field image of TNBC tissue, (F & G) Raman image of the same area w.r.t 440 and 580 nm spectral fingerprint, (H) Average spectrum from the scan.

FIG. 8b. Simultaneous detection of three biomarkers: SERS images from the breast cancer tissues incubated with AuNP@SDL@PEG@antiER and AuNP@CV@PEG@antiHER2 and AuNP@MBA@PEG@antiPR were collected. (A) Bright field image of ER+/PR+/HER2+ tissue, (B) Raman spectrum from image scan showing simultaneous peaks for HER2, ER and PR, (C) Raman image w.r.t 440 cm−1, spectral fingerprint, (D) Raman image w.r.t 580 cm−1 spectral fingerprint, (E) Raman image w.r.t 1080 cm−1, spectral fingerprint [C, D, E are from the same area of bright field image (A)].

FIG. 9. SERS analysis for Ki67 expression in TNBC tissue sample: TNBC samples were incubated with AuNP@Py L Et@antiKi67 for 30 min and SERS analysis was performed after thorough washing.

FIG. 10. HER-2 Grading of tissue samples with SERS analysis: a) Immunohistochemical analysis, b) SERS images from image scan and c) representative spectra from different HER2 grades.

### DETAILED DESCRIPTION OF THE INVENTION

The present invention is focused on simultaneous detection of multiple biomarkers in a breast tissue sample based on SERS-nanotags using Raman fingerprint analysis. A nanoparticle probe comprising gold or silver nanoparticles having size in the range of 40-50 nm anchored with a Raman reporter molecule and encapsulated with a biocompatible polymer is conjugated with a breast cancer specific antibody which is transformed into a SERS-nanotag. Further, these SERS-nanotag conjugated to target specific antibodies raised against a biomarker selected from the group consisting of Estrogen Receptor (ER), Progesterone Receptor (PR), Human Epidermal Growth Factor Receptor 2 (HER2) and Ki67are used to validate the simultaneous recognition capabilities in paraffin embedded breast cancer tissue samples.

A diagnostic kit comprising the SERS-nanotag enables simultaneous detection of multiple biomarkers in a breast tissue sample with a highly sensitive and specific Raman peak of the Raman reporter attached to the nanoparticle which corresponds to the presence of respective antibody attached to the same nanoparticle.

An embodiment of the present invention provides a SERS-nanotagcomprising:
i. gold nanoparticles having size in the range of 40-50 nm;
ii. an encapsulating agent;
iii. a Raman reporter molecule; and
iv. an antibody

In another embodiment of the present invention, there is provided a SERS-nanotag, wherein the encapsulating agent is selected from the group consisting of a polysaccharide, polyethylene glycol, and serum albumin.

In still another embodiment of the present invention, there is provided a SERS-nanotag, wherein the polysaccharide is selected from the group consisting of chitosan, and hyaluronic acid.

In yet another embodiment of the present invention, there is provided a SERS-nanotag, wherein the encapsulating agent is polyethylene glycol

In another embodiment of the present invention, there is provided a SERS-nanotag, wherein the Raman reporter molecule is selected from the group consisting of cyanine dilipoic acid (Cy7DLA), hemicyaninecarbaldehyde (HCC), Pyryliniumhexylamine (PHA), Squaraine di-lipoic acid (SDL), Pyrenelipidene ethyl quartanised (Py L Et), crystal violet (CV) and Mercapto benzoic acid (MBA).

In still another embodiment of the present invention, there is provided a SERS-nanotag, wherein the antibody is a monoclonal or a polyclonal antibody.

In yet another embodiment of the present invention, there is provided a SERS-nanotag, wherein the antibody is a monoclonal antibody.

In still another embodiment of the present invention, there is provided a SERS-nanotag, wherein the antibody is a polyclonal antibody.

In an embodiment of the present invention, there is provided a SERS-nanotag, wherein the antibody is raised against a biomarker selected from the group consisting of Estrogen Receptor (ER), Progesterone Receptor (PR), Human Epidermal Growth Factor Receptor 2 (HER2) and Ki67.

An embodiment of the present invention provides a process for synthesis of the SERS-nanotag comprising the steps of:
a. providing gold nanoparticles having size in the range of 40-50 nm in a solution;
b. concentrating the gold nanoparticles of step (a) by centrifugation at 6000 rpm for 30 minutes followed by addition of 0.05% TWEEN 20 to obtain a stabilized concentrated gold nanoparticle solution;
c. adding a Raman reporter molecule to the concentrated gold nanoparticle solution obtained in step (b) and incubating for 30 minutes followed by addition of an encapsulating agent and incubating for 3 hours to obtain a biocompatible gold nanoparticle solution;
d. concentrating the biocompatible gold nanoparticle solution obtained in step (c) by centrifugation at 10,000 rpm for 10 minutes and removing excess encapsulating agent to obtain a solution;
e. re-suspending the solution obtained in step (d) in a buffer and adding (1-Ethyl-3-(3-dimethylaminopropyl) carbodiimide and sulfo-NHS to obtain a reaction mixture;
f. incubating the reaction mixture obtained in step (e) for 30 minutes, centrifuging and re-suspending in the buffer;
g. adding an antibody to the reaction mixture of step (f) and incubating in a shaker incubator;
h. centrifuging the reaction mixture after incubation and re-suspending in the buffer to obtain the SERS-nanotag.

In an embodiment of the present invention, there is provided a process for synthesis of the SERS-nanotag, wherein the gold nanoparticles are in a concentration in the range of 7×10⁹ to 4×10¹⁰ particles/mL

In another embodiment of the present invention, there is provided a process for synthesis of the SERS-nanotag, wherein the Raman reporter molecule is in a concentration in the range of 0.5 to 100 μM.

In yet another embodiment of the present invention, there is provided a process for synthesis of the SERS-nanotag, wherein the antibody is in a concentration in the range of 2 to 20 μg.

In an embodiment of the present invention, there is provided a process for synthesis of the SERS-nanotag, wherein the Raman reporter molecule is selected from the group consisting of cyanine dilipoic acid (Cy7DLA), hemicyaninecarbaldehyde (HCC), Pyryliniumhexylamine (PHA), Squaraine di-lipoic acid (SDL), Pyrenelipidene ethyl quartanised (Py L Et), crystal violet (CV) and Mercapto benzoic acid (MBA).

In another embodiment of the present invention, there is provided a process for synthesis of the SERS-nanotag, wherein the encapsulating agent is selected from the group consisting of a polysaccharide, polyethylene glycol, and serum albumin

In yet another embodiment of the present invention, there is provided a process for synthesis of the SERS-nanotag, wherein the buffer is selected from the group consisting of MES buffer, Phosphate buffer and Tris buffer.

In an embodiment of the present invention, there is provided a process for synthesis of the SERS-nanotag, wherein the antibody is raised against a biomarker selected from the group consisting of Estrogen Receptor (ER), Progesterone Receptor (PR), Human Epidermal Growth Factor Receptor 2 (HER2) and Ki67.

Another embodiment of the present invention provides a diagnostic kit for detection of breast cancer biomarker comprising:
I. the SERS-nanotag;
II. xylene;
III. absolute ethanol;
IV. citrate buffer;
V. phosphate buffer saline;
VI. bovine serum albumin; and
VII. instructions manual.

Yet another embodiment of the present invention provides a method for detecting breast cancer biomarker in a tissue sample comprising the steps of:
(i) taking a paraffin embedded formalin fixed tissue sample;
(ii) washing the sample with xylene;
(iii) washing the sample of step (ii) with absolute ethanol followed by washing with 95% ethanol followed by washing with 70% ethanol and then with 50% ethanol to obtain a washed tissue sample;
(iv) treating the washed tissue sample of step (iii) with citrate buffer to obtain a treated tissue sample;
(v) incubating the treated tissue sample of step (iv) with bovine serum albumin and washing with phosphate buffer saline;
(vi) incubating the tissue sample of step (v) with the SERS-nanotag for 30 minutes and washing;
(vii) performing Raman spectroscopy on the tissue sample of step (vi) to take signature peaks; and
(viii) analyzing the peaks to confirm the presence of breast cancer biomarker.

In still another embodiment of the present invention, there is provided a method for detecting breast cancer biomarker in a tissue sample, wherein the breast cancer biomarker is selected from the group consisting of Estrogen receptor (ER), Progesterone receptor (PR), Human Epidermal Growth Factor Receptor 2 (HER2) and Ki67.

The aim of the present invention is to detect multiple biomarkers in a breast tissue sample by SERS based diagnostic platform in a simultaneous detection mode using SERS-nanotags and confirm the presence or absence of the biomarkers much faster with high precision level as compared to analysis with current gold standards. The present invention provides a SERS based simultaneous diagnostic kit having a SERS-nanotag comprising a nanoparticle, a biocompatible polymer, a Raman reporter (RR) molecule and an antibody raised against breast cancer biomarkers selected from the group consisting of Estrogen receptor (ER), Progesterone receptor (PR), Human Epidermal Growth Factor Receptor 2 (HER2) and Ki67.

The nanoparticle can be colloidal gold nanoparticles (AuNPs), colloidal silver nanoparticles (AgNPs), gold coated silver nanoparticles (Au@AgNPs) or gold (Au)/silver (Ag) coated glass slide. In a preferred embodiment of the present invention, the nanoparticles are AuNPs having size in the range of 40-50 nm. These gold nanoparticles are then encapsulated in a biocompatible polymer which is selected from the group consisting of a polysaccharide, polyethylene glycol, and serum albumin Both, in-house synthesized and commercially available Raman reporters are used as a signature Raman peaks to obtain simultaneous detection. The in-house Raman reporter (RR) molecules used in the present invention are Cy7DLA (cyanine dilipoic acid), HCC (hemicyaninecarbaldehyde), PHA (Pyryliniumhexylamine), SDL (Squaraine di-lipoic acid), Py L Et (Pyrenelipidene ethyl quartanised), and commercially available Raman reporters include CV (crystal violet) and MBA (Mercapto benzoic acid). The Raman reporter molecules are coupled to the commercially purchased antibodies raised against biomarkers i.e. ER, PR, HER2, and Ki67, to formulate the SERS-nanotags for detection of breast cancer biomarkers i.e. ER, PR, HER2, and Ki67.

The present invention also co-relates and validates the Raman fingerprint from the Raman reporter molecule with-respect-to biomarker in a breast tissue sample in a simultaneous

Raman fingerprint. This programme was approved by the local Ethics Committee and prior to specimen collection; all patients had signed informed consent forms. Pathologically confirmed breast cancer tissues with different ER, PR, HER2, Ki67 status were collected from Regional Cancer Centre (RCC), Trivandrum, Kerala, India. A separate bit of tumor tissue from each subject was paraffin embedded and 4 micron sections were obtained. Sections were selected after confirming the presence of tumor in Haematoxylin and eosin stained samples. Paraffin wax was removed by rinsing in xylene followed by different grades of alcohol and antigen retrieval was performed prior to the incubation with SERS-nanotag. SERS spectral analysis was carried out using with diode laser of 633/785 nm laser excitation source with spectrograph grating 600 gr/mm using maximum 1-20 sec integration time and around 1-15 accumulations.

### EXAMPLES

The following examples are given by way of illustration of the present invention and therefore should not be construed to limit the scope of the present invention.

### Example 1

### Preparation of SERS Substrate

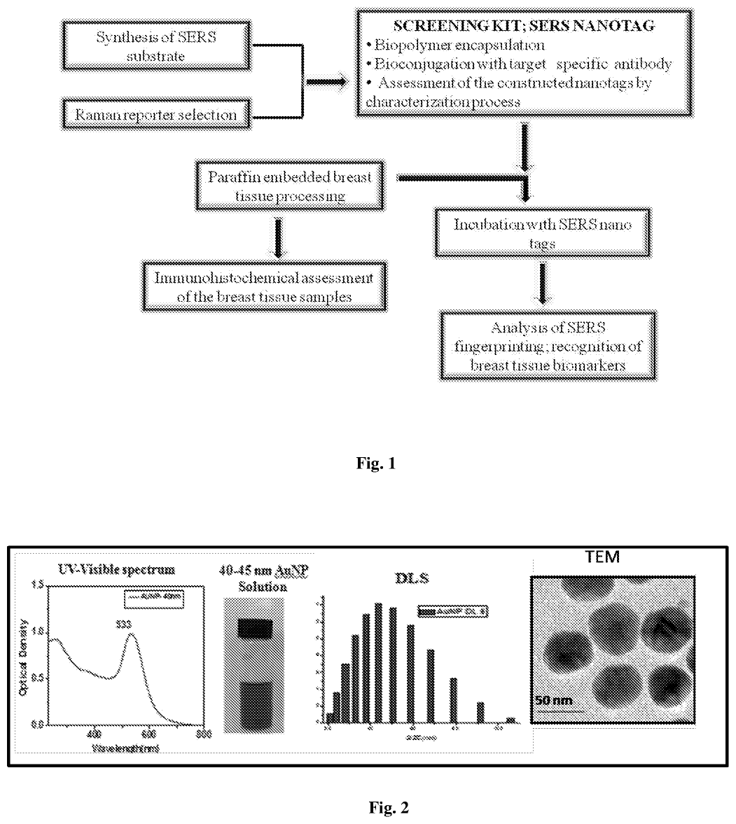

Colloidal gold nanoparticles (AuNPs, size around 40-50 nm) were prepared by citrate reduction method [Kim Ling, J., Maier, M., Okenve, B., Kotaidis, V., Ballot, H. and Plech, A., 2006. Turkevich method for gold nanoparticle synthesis revisited. The Journal of Physical Chemistry B, 110(32), pp. 15700-15707]. The characterization of the synthesized nanoparticles was done through UV-VIS spectroscopy, Dynamic Light Scattering (DLS) and High Resolution Transmission Electron Microscopy (HRTEM). FIG. 2 shows the synthesis and characterization of AuNPs/AgNPs. The size was approximately 40-50 nm which served as an optimal SERS substrate in order to get maximum enhancement.

### Example 2

### Synthesis of Nanotag: AuNP@PEG@CV

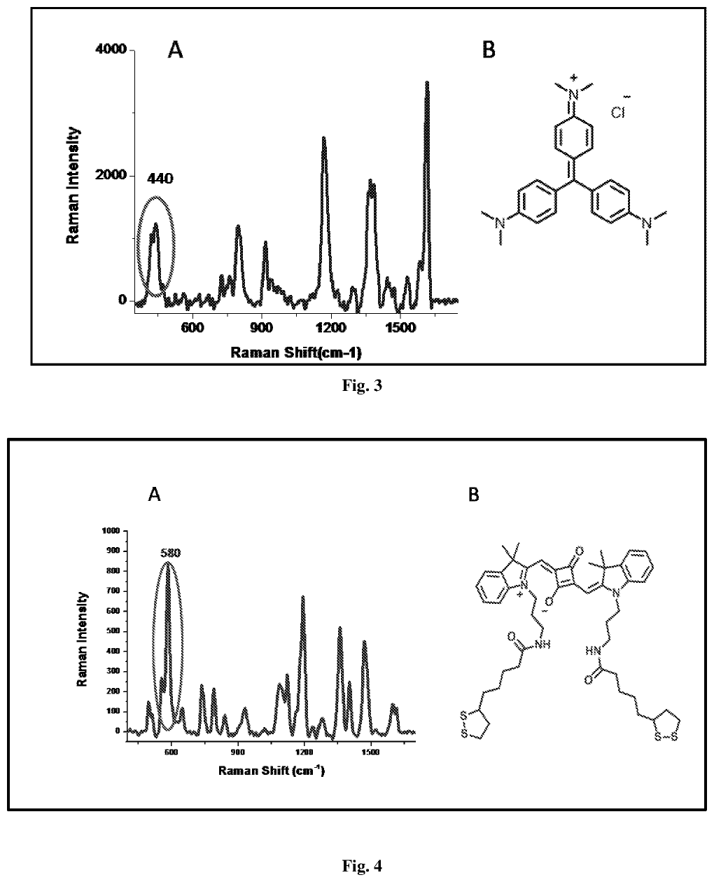

The gold nanoparticles of size 40-45 nm were concentrated from 25 mL to 3.6 mL by centrifugation at 6000 rpm, for 30 minutes. To this, 0.05% of TWEEN 20 was added for stabilizing the gold nanoparticles and vortexed for few minutes. Then, ˜400 μL of 80 μM Raman reporter1 (crystal violet (CV) in dimethyl sulfoxide (DMSO)) was added and incubated for half an hour. For making the tag biocompatible, 45 μL SH-PEG-COOH was added and incubated for 10 minutes. To this solution, 275 μL SH-PEG-OCH₃ was added and further incubated for 3 hrs. Then, the solution was concentrated to 1 mL by centrifugation at 10,000 rpm for 10 minutes. Excess PEG was removed by centrifuging the solution again at 10,000 rpm for 10 minutes. Finally, the solution was re-suspended in milliQ water to obtain AuNP@PEG@CV. FIG. 3 shows gold nanoparticle with CV as the Raman reporter: A) Raman spectrum of PEGylated AuNP with CV as the reporter and B) Structure of CV.

### Example 3

### Synthesis of SERS-Nanotag: AuNP@PEG@CV@AntiHER2

Anti-HER2 (Rabbit Monoclonal antibody; ABCAM) for the biomarker HER2 was purified using 3 KDa centrifugal filters. PEG encapsulated nanoparticles obtained in example 2 (1-1.5 mL) were centrifuged at 8000 rpm for 15 minutes and re-suspended in −500 μL MES buffer (HIMEDIA) (50 mM, pH 6.1). 5 μL of freshly prepared EDC (SIGMA-ALDRICH) (1-Ethyl-3-(3-dimethylaminopropyl) carbodiimide, 250 mM) was added and after a few minutes gap, 6 μL of freshly prepared sulfo-NHS (ALDRICH CHEMISTRY) (N-hydroxysuccinimide, 250 mM) was also added. After incubating the reaction mixture for 30 minutes, the reaction mixture was centrifuged at 10000 rpm for 10 minutes and re-suspended in 500 μL MES buffer. Then, 4 μg antibody was added to that and incubated in a shaker incubator for 2 hrs at room temperature and kept for overnight incubation at 4° C. After that, the mixture was centrifuged at 10000 rpm for 10 minutes and finally re-suspended in fresh 500 μLMES to obtain the antibody conjugated SER-nanotag AuNP@PEG@CV@Anti HER2.

### Example 4

### Synthesis of Nanotag: AuNP@PEG@SDL

The gold nanoparticles of size 40-45 nm were concentrated from 7.2 mL to 1 mL by centrifugation at 6000 rpm, for 30 minutes). 0.05% of TWEEN 20 was added for stabilizing the gold nanoparticles and vortexed for few minutes. Then, 37 μL of 100 μM Raman reporter 2 (Squaraine di-lipoic acid (SDL) in dimethyl sulfoxide) and 262.5 μL milliQ water was added and incubated for 10 minutes. For making the tag biocompatible, 62 μL SH-PEG-COOH was added and incubated for 10 minutes. To this solution, 368 μL SH-PEG-OCH3 was added and further incubated for 3 hrs. Then, the solution was concentrated to 1 mL by centrifugation at 10,000 rpm for 10 minutes. Excess PEG was removed by centrifuging the solution again at 10,000 rpm for 10 minutes. Finally, the solution was re-suspended in milliQ water to obtain AuNP@PEG@SDL. FIG. 4 shows gold nanoparticle with SDL as the Raman reporter: A) Raman spectrum of PEGylated AuNP@SDL and B) structure of SDL.

### Example 5

### Synthesis of SERS-Nanotag: AuNP@PEG@SDL@Anti ER

Anti-ER (Rabbit Monoclonal antibody; ABCAM) for the biomarker ER was purified and conjugated by EDC-NHS coupling as described in example 3 (procedure for HER2 conjugation). Here also, 4 μg antibody was added to the PEGylated AuNPs obtained in example 4 to obtain antibody conjugated SERS-nanotag: AuNP@PEG@SDL@Anti ER.

### Example 6

### Synthesis of Nanotag: AuNP@PEG@MBA

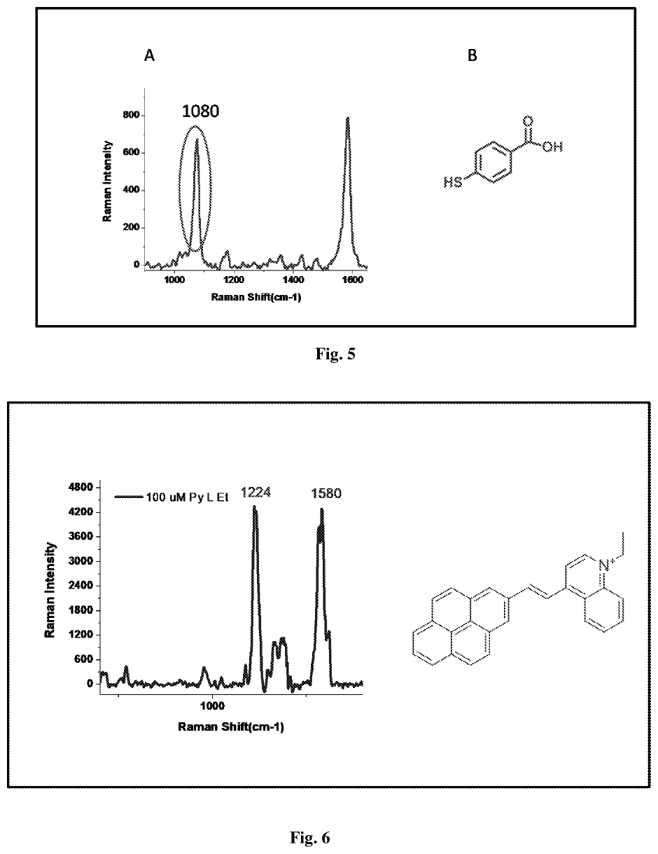

The gold nanoparticles of size 40-45 nm were concentrated from 25 mL to 3.6 mL by centrifugation at 6000 rpm, for 30 minutes. To this, 0.05% of TWEEN 20 was added for stabilizing the gold nanoparticles and vortexed for few minutes. Then, 400 μL of 200 μM Raman reporter 3 Mercapto benzoic acid (MBA) was added and incubated for half an hour. For making the tag biocompatible, 45 μL SH-PEG-COOH was added and incubated for 10 minutes. To this solution, 275 μL SH-PEG-OCH3 was added and further incubated for 3 hrs. Then, the solution was concentrated to 1 mL by centrifugation at 10,000 rpm for 10 minutes. Excess PEG was removed by centrifuging the solution again at 10,000 rpm for 10 minutes. Finally, the solution was re-suspended in milliQ water to obtain SERS-nanotag: AuNP@PEG@MBA. FIG. 5 shows gold nanoparticle with MBA as the Raman reporter: A) Raman spectrum of AuNP@PEG@MBA and B) Structure of MBA.

### Example 7

### Synthesis of SERS-Nanotag: AuNP@PEG@MBA@Anti-PR

Anti-PR (Rabbit Monoclonal antibody; ABCAM) for the biomarker PR was purified and conjugated to the AuNP@PEG@MBA obtained in example 6 by the procedure described in example 3 to obtain antibody conjugated SERS-nanotag: AuNP@PEG@MBA@Anti-PR.

### Example 8

### Synthesis of Nanotag: AuNP@PEG@Py L Et

The gold nanoparticles of size 40-45 nm were concentrated from 25 mL to 3.6 mL by centrifugation at 6000 rpm, for 30 minutes. To this, 0.05% of TWEEN 20 was added for stabilizing the gold nanoparticles and vortexed for few minutes. Then, 400 μL of 100 μM Raman reporter 4 Pyrenelipidene ethyl quartanised (Py L Et) was added and incubated for half an hour. For making the tag biocompatible, 45 μL SH-PEG-COOH was added and incubated for 10 minutes. To this solution, 275 μL SH-PEG-OCH3 was added and further incubated for 3 hrs. Then, the solution was concentrated to 1 mL by centrifugation at 10,000 rpm for 10 minutes. Excess PEG was removed by centrifuging the solution again at 10,000 rpm for 10 minutes. Finally, the solution was re-suspended in milliQ water to obtain SERS nanotag: AuNP@PEG@Py L Et. FIG. 6 shows gold nanoparticle with Py L Et as the Raman reporter: A) Raman spectrum of AuNP@PEG@Py L Et and B) Structure of Py L Et.

### Example 9

### Synthesis of SERS-Nanotag AuNP@PEG@Py L Et@Anti-Ki67

Anti-Ki67(ABCAM, Mouse monoclonal antibody) for the biomarker Ki67 was purified and conjugated to the AuNP@PEG@Py L Et (4 μg) obtained in example 8 by the procedure described in example 3 to obtain antibody conjugated SERS-nanotag AuNP@PEG@Py L Et@Anti-Ki67.

### Example 10

### Single Cell Spectral Analysis From Paraffin Embedded Tissue Sample

Breast cancer tissue samples having various biomarker expression status were collected from Regional Cancer Centre, Trivandrum, Kerala, India for SERS analysis. Ethical approval for the same was obtained from the consigned authorities prior to the experiments.

### Optimized Processing of Tissue

Prior to spectral analysis, tissue processing was carried out by the following standardized steps for the paraffin embedded formalin fixed tissues.
A) Deparaffinization in Xylene: The paraffin embedded formalin fixed tissues were washed with xylene for 3 times, 8 minutes each.
B) Hydration By Graded Alcohol: Then the formalin fixed tissues were washed with absolute ethanol for 2 times, 5 minutes each; Then the formalin fixed tissues were washed with 95% Ethanol for 3 minutes, then washed with 70% Ethanol for 3 Minutes, then washed with 50% Ethanol for 3 Minutes, then washed with distilled water for 5 minutes.
C) Antigen Retrieval: The washed formalin fixed tissues were treated with 10 Mm citrate buffer (pH 6.1) at 500-700 W for 10 minutes in a microwave oven and then kept for 1-5 minutes rest. Then, more volume of citrate buffer was added to the tissues and again heated for 5-10 minutes at 500-700 W. The slides of formalin fixed tissues were allowed to cool at room temperature for 15-20 minutes and then immersed in de-ionized water for 15-20 minutes at room temperature.
D) Blocking With BSA: The fixed tissues were incubated with 3% BSA in PBS at room temperature and then washed three times with PBS.
E) Incubation With SERS Nanotag: The fixed tissues were incubated with antibody conjugated SERS nanotags for 30 minutes in a humid chamber and then washed 3-5 times with PBS. Raman spectra and Raman images were acquired under 633/785 nm laser by placing the samples under 10× or 20× on objective of Raman microscope. Confocal Raman microscope (WITec, Germany) with Peltier cooled charge-coupled device detector unit was used for the analysis. The samples were excited using a 633/785 nm laser with 10 mW-30 mW power, and Raman spectra were collected in the region of 300-2000 cm⁻¹ with a resolution of 1 cm⁻¹ and an integration time of 2-10 seconds and 3-5 accumulations. Prior to each analysis, calibration was done with a silicon standard (Raman peak at 520 cm⁻¹). Data processing was performed using WITec Project Plus (v5.2) software package. Raman imaging was performed for an area of 150 -175 μm×150 -175 μm area with an integration time of 0.01 to 0.1 seconds integration time and 100-150 lines and points per image.

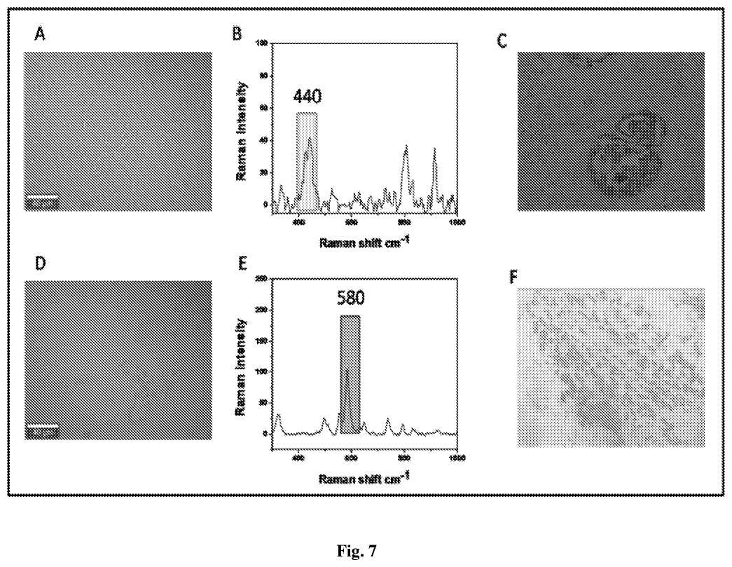

FIG. 7 shows SERS single spectral analysis of one biomarker: (A) Bright field image of ER/PR negative HER-2 positive tissue; (B) SERS finger print from the same tissue incubated with AuNP@PEG@CV@Anti-HER-2+AuNP@PEG@SDL@Anti-ER, shows the 440 cm⁻¹ marker peak of CV which was used as the corresponding Raman reporter for HER-2 biomarker detection. As the sample analyzed was HER-2 positive and ER negative tissue, the spectral pattern obtained was also lacking the 580 cm⁻¹ marker peak of SDL correspond to ER biomarker; (C) Immunohistogram of the same tissue stained for HER-2 showing the deeply stained HER-2 positive cells; (D) Bright field image of ER/PR positive HER-2 negative tissue; (E) SERS spectra from the same tissue incubated with AuNP@PEG@CV@Anti-HER-2+AuNP@PEG@SDL@Anti-ER, spectral pattern from tissue sample showing the 580 cm⁻¹ marker peak of SDL but not the 440 cm⁻¹ of CV denotes the ER positive and HER-2 negative expression status; (F) Immunohistogram of the same tissue stained for ER showing the ER positive cells.

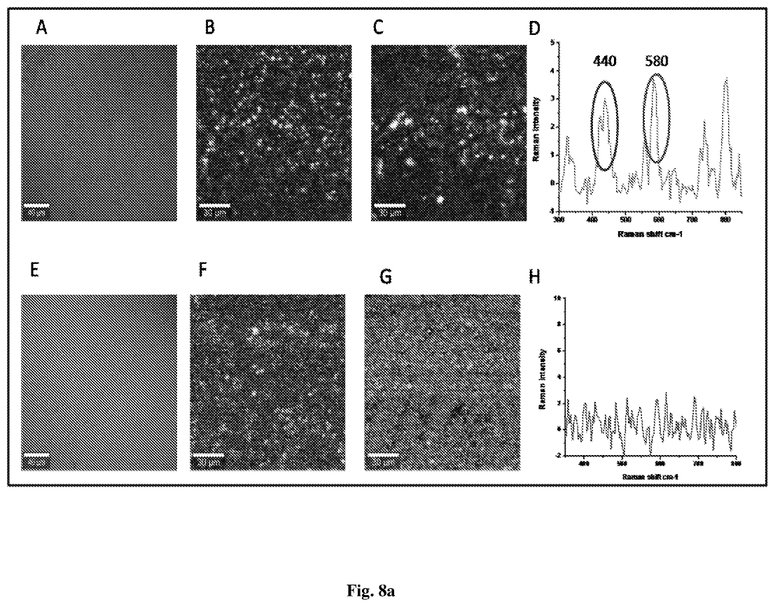

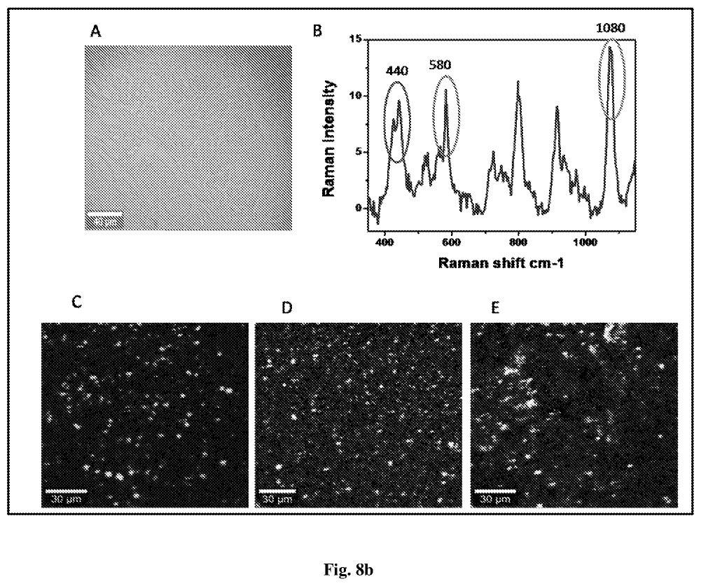

FIG. 8 shows the SERS imaging of breast cancer tissues. FIG. 8a shows simultaneous detection of two biomarkers: SERS images from the breast cancer tissues incubated with AuNP@SDL@PEG@antiER and AuNP@CV@PEG@antiHER2 were taken; (A) Bright field image of ER+/HER2+ tissue; (B) Raman image w.r.t 440 nm, spectral fingerprint with bright spots showing the HER-2 positive cells; (C) Raman image w.r.t 580 nm spectral fingerprint with bright spots denoting the ER positive cells; (D) Average spectrum from the scan presenting the 440 cm⁻¹ and 580 cm⁻¹ peaks of CV and SDL corresponding to HER-2 and ER biomarkers respectively portraying the ER and HER-2 positivity [B, C, and D, are from the same area of bright field image (A)]; (E) Bright field image of TNBC tissue; (F & G) Raman image of the same area w.r.t 440 and 580 nm spectral fingerprint; (H) Average spectrum from the scan without CV and SDL peaks depicts the absence of biomarkers.

FIG. 8b shows the simultaneous detection of three biomarkers: SERS images from the breast cancer tissues incubated with AuNP@SDL@PEG@antiER and AuNP@CV@PEG@antiHER2 and AuNP@MBA@PEG@antiPR were collected: (A) bright field image of ER+/PR+/HER2+ tissue; (B) Raman spectrum from image scan showing simultaneous peaks for HER2 (440 cm⁻¹ peak of CV), ER (580 cm⁻¹ peak of SDL) and PR (1080 cm⁻¹ peak of MBA) showing the presence of all the three biomarkers; (C) Raman image w.r.t 440 cm−1, spectral fingerprint; (D) Raman image w.r.t 580 cm−1 spectral fingerprint; (E) Raman image w.r.t 1080 cm−1, spectral fingerprint [C, D, E are from the same area of bright field image (A)].

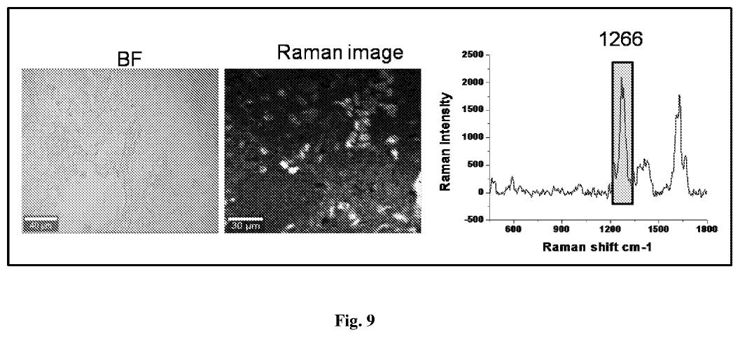

FIG. 9 shows the SERS analysis for Ki67 expression in TNBC tissue sample: TNBC samples were incubated with AuNP@Py L Et@antiKi67 for 30 min and SERS analysis was performed after thorough washing. Spectral data shows the Py L Et peak and the Raman image with bright spots shows the Ki67 positive cells denoting the Ki67 biomarker expression in the tissue sample.

The present diagnostic strategy is able to detect single, duel and triple biomarkers in breast cancer tissue sample accurately. FIG. 7 shows precisely the presence of single biomarker i.e. HER-2 and ER in respective tissue sample. In FIG. 8, dual biomarker i.e. HER-2 and ER were detected in a single tissue sample whereas in TNBC sample, absence of any reporter peaks were observed. Similarly, ER, PR and HER-2 biomarkers were simultaneously detected from ER+/PR+/HER-2+ tissue sample and Ki67 abundance in TNBC sample.

### Example 11

### HER-2 Grading by SERS

Breast cancer tissue samples with different levels of HER-2 expression were collected from Regional Cancer Centre (RCC), Trivandrum, Kerala, India. Tissues were analyzed by immunohistochemistry to confirm the grading by a pathologist. Samples were processed as mentioned in above examples. For HER-2 grading, AuNP@CV@HER-2 was added to the tissue samples and incubated for 30 min and washed thoroughly to take the signature peaks from SERS analysis. Spectra were collected by spectral accumulation and also through image scanning. All the tissue processing procedure was as early described and the SERS analysis was performed with 633 nm laser, under 10× objective of confocal Raman microscope. For single spectral analysis, integration time used was 3-5 seconds with 2-5 accumulations using 10 mW power. Image scanning was performed for 150×150 nm area with 0.01 seconds integration time. Average CCD counts were taken for comparison of different HER-2 graded samples.

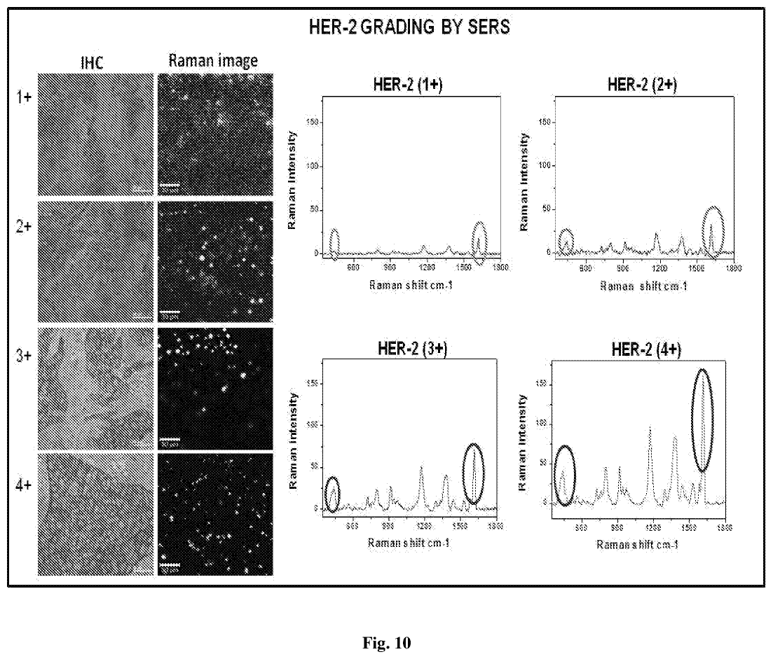

FIG. 10 shows the HER-2 Grading of tissue samples with SERS analysis: (a) Immunohistochemical analysis showing the increased expression of HER-2 from 1+ to 4+ sample; (b) SERS images from image scan with increased number of bright spots in the images as the HER-2 expression increases; and (c) representative spectra from different HER2 grades. Table 1 provides the average CCD counts for the signature peaks acquired from different HER2 grades.

TABLE 1

| HER-2 Grade | Avrg CCD @ 440 | Avrg CCD@1615 |
|---|---|---|
| 1+ | 3 | 18 |
| 2+ | 15 | 32 |
| 3+ | 24 | 65 |
| 4+ | 40 | 1150 |

HER-2 grading is an important aspect during the selection of treatment regimens for breast cancer patients as HER-2 over expression, i.e., 3+ and above are considered as HER-2 positive whereas 2+ expression is considered as borderline. Here, tissue samples with varying HER-2 expression levels (1+ to 4+) were analyzed by single spectral obtained from the Raman mapping providing the average spectra. Both the analysis showed an increase in spectral intensity of Raman reporter CV in accordance with the Her-2 expression levels. Table 1 denotes the CCD counts obtained from the Raman image analysis corresponding to the 440 and 1615 cm⁻¹ peaks of CV where both the peak intensities were found to be increasing from 3 to 40 w.r.t to peak at 440 cm⁻¹ and 18 to 150 w.r.t. peak at 1615 cm⁻¹ respectively as the HER-2 expression levels increases from 1+ to 4+. Thus, the present Her-2 grading by Raman spectral analysis can be utilized as a complementary technique to IHC to confirm the HER-2 grading.

### Comparison of the Technique of the present Invention with IHC

Immunohistochemistry (IHC) is the existing gold standard method for detection of breast cancer biomarkers in formalin fixed paraffin embedded tissue samples. Table 2 provided below compares both the techniques in terms of specificity, easiness and time required for sample processing and analysis.

TABLE 2

| | | SERS Diagnostic kit of |
|---|---|---|
| Parameters | IHC | the present invention |
| Mutiplexing | Very difficult. No standard | Easily to analyze in |
| Analysis | method. | single tissue sample |
| Type of | Highly Subjective (Inter | Objective; semi- |
| analysis | observer variation) | quantitative analysis is |
| | | possible for Her-2 |
| | | grading |
| Time | 4-27 hrs | 4-5 hrs |
| required | | |
| for sample | | |
| preparation | | |
| Time | 0.5 hr for single biomarker | 0.5-1 hr irrespective of |
| required | | single or more biomarkers |
| for analysis | | |
| Specificity | >95% | Nearly 90% for single |
| | | biomarker., 80-85% |
| | | multiplexing analysis |
| False | 0-36% may happen due to | 10-30% may happen due to |
| positive | nonspecific binding of horse | the nonspecific binding of |
| results | radish peroxidase (HRP) | nanoparticles |
| | conjugated secondary | |
| | antibody | |
| | (Nuovo, G., 2016. False- | |
| | positive results in diagnostic | |
| | immunohistochemistry are | |
| | related to horseradish | |
| | peroxidase conjugates in | |
| | commercially available | |
| | assays. Annals of | |
| | Diagnostic Pathology, | |
| | 25, pp.. 54-59.) | |
| False | 0-10% due to low levels of | More sensitive technique, |
| negative | biomarker in the sample, poor | Approximately 0-8% may |
| result | tissue fixation, problems with | happen due to poor |
| | the tissue processing and | fixation, tissue processing |
| | antigen retrieval steps etc | and antigen retrieval steps |
| | (True, L.D. 2008. Quality | |
| | control in molecular | |
| | immunohistochemistry. | |
| | Histochemistry and Cell | |
| | Biolo, 130, pp. 473-480.) | |

### Advantages of the Invention

1. Development of diagnostic screening kit for accurate detection of a diseases is a challenging task in biomedical research. In the field of bio-imaging and diagnostics, SERS has emerged as a highly sensitive and promising technique for detection of biological and chemical molecules which are adsorbed on nano roughened metallic surfaces like gold or silver.
2. The present invention provides a SERS-nanotag comprising colloidal AuNPs, a Raman reporter molecule, a biocompatible polymer and an antibody raised against a biomarker selected from the group consisting of ER, PR, HER2 and Ki67 for specific simultaneous detection of ER, PR, HER2, and Ki67. The preparative steps are critically optimized for highly selective detection of clinically relevant biomarkers only from breast tissue samples.
3. In the present invention, a tissue processing step and an antigen retrieval has been incorporated with an easy and straight forward way to remove the paraffin wax and unmask the antigens from the paraffin embedded breast tissue.
4. In the present invention, SERS analysis i.e. scanning, and imaging of the SERS-nanotag is performed to gather the information from maximum locations in order to know the abundance of biomarkers in the breast tissue sample.
5. In the present invention, a SERS intensity based semi-quantitative system for HER-2 gradation has been provided using the SERS-nanotag since the over expression of HER-2 (2+ and above from immunohistochemistry grading) is considered by the clinicians to judge the samples as positive.
6. The present invention demonstrates an ultrasensitive simultaneous detection modality based on SERS-nanotags which aims novelty technical advancement over the existing technology.
7. Simultaneous recognition of breast cancer biomarkers ER, PR, HER2, and Ki67 expression in a single detection mode with a single laser utilizing respective antibody conjugated SERS-nanotag of the present invention is termed as SERS based immunoassays.
8. Simultaneous detection modality of the present invention is achieved by initial validation in paraffin embedded breast cancer tissue samples. By evaluation of SERS spectral analysis of the emission from SERS-nanotag, ER, PR, HER2 and Ki67 status from the tissue sample is confirmed which definitely propagates into treatment management with high precision, minimum assay time, and in a cost effective manner
9. The nanotag of the present invention is highly accurate and there is very low possibility of false positive and false negative results.

1-18. (canceled)

19. A SERS-nanotag comprising:
gold nanoparticles having a size of from 40 nm to 50 nm;
an encapsulating agent;
a Raman reporter molecule; and
an antibody,
wherein:
the Raman reporter molecule is selected from the group consisting of cyanine dilipoic acid (Cy7DLA), hemicyaninecarbaldehyde (HCC), pyryliniumhexylamine (PHA), squaraine di-lipoic acid (SDL), pyrenelipidene ethyl quartanised (Py L Et), and mercaptobenzoic acid (MBA);
the antibody is raised against a biomarker selected from the group consisting of Estrogen Receptor (ER), Progesterone Receptor (PR), Human Epidermal Growth Factor Receptor 2 (HER2), and Ki67; and
the SERS-nanotag detects multiple biomarkers simultaneously.

20. The SERS-nanotag of claim 19, wherein the encapsulating agent is selected from the group consisting of polysaccharides, polyethylene glycol, and serum albumin.

21. The SERS-nanotag of claim 20, wherein the encapsulating agent comprises a polysaccharide selected from the group consisting of chitosan and hyaluronic acid.

22. The SERS-nanotag of claim 20, wherein the encapsulating agent is polyethylene glycol.

23. The SERS-nanotag of claim 19, wherein the antibody is a monoclonal antibody or a polyclonal antibody.

24. A process for synthesizing the SERS-nanotag according to claim 19, the process comprising:
(a) providing gold nanoparticles having sizes of from 40 nm to 50 nm in a solution;
(b) concentrating the gold nanoparticles of (a) by centrifugation at 6000 rpm for 30 minutes followed by adding 0.05% TWEEN 20 to obtain a stabilized concentrated gold nanoparticle solution;
(c) adding a Raman reporter molecule to the stabilized concentrated gold nanoparticle solution obtained in (b) and incubating for 30 minutes followed by adding an encapsulating agent and incubating for 3 hours to 4 hours to obtain a biocompatible gold nanoparticle solution;
(d) concentrating the biocompatible gold nanoparticle solution obtained in (c) by centrifugation at 10,000 rpm for 10 minutes and removing excess encapsulating agent to obtain a solution;
(e) re-suspending the solution obtained in (d) in a buffer and adding 1-Ethyl-3-(3-dimethylaminopropyl) carbodiimide and sulfo-NHS to obtain a reaction mixture;
incubating the reaction mixture obtained in (e) for 30 minutes, centrifuging and re-suspending in the buffer;
(g) adding an antibody to the reaction mixture after (f) and incubating in a shaker incubator; and
(h) centrifuging the reaction mixture after incubation, and re-suspending in the buffer to obtain the SERS-nanotag.

25. The process of claim 24, wherein the gold nanoparticle are in a concentration of from 7×10⁹ particles/mL to 4×10¹⁰ particles/mL.

26. The process of claim 24, wherein the Raman reporter molecule is in a concentration of from 0.5 μM to 100 μM.

27. The process of claim 24, wherein the antibody is in a concentration of from 2 μg/mL to 20 μg/mL.

28. The process of claim 24, wherein the Raman reporter molecule is selected from the group consisting of cyanine dilipoic acid (Cy7DLA), hemicyaninecarbaldehyde (HCC), pyryliniumhexylamine (PHA), squaraine di-lipoic acid (SDL), pyrenelipidene ethyl quartanised (Py L Et), crystal violet (CV), and mercaptobenzoic acid (MBA).

29. The process of claim 24, wherein the encapsulating agent is selected from the group consisting of polysaccharides, polyethylene glycol, and serum albumin.

30. The process of claim 24, wherein the buffer is selected from the group consisting of MES buffer, phosphate buffer, and Tris buffer.

31. The process of claim 24, wherein the antibody is raised against a biomarker selected from the group consisting of Estrogen Receptor (ER), Progesterone Receptor (PR), Human Epidermal Growth Factor Receptor 2 (HER2), and Ki67.

32. A diagnostic kit for detection of a breast cancer biomarker with the SERS-nanotag according to claim 19, the diagnostic kit comprising:
the SERS-nanotag;
xylene;
absolute ethanol;
citrate buffer;
phosphate buffer saline;
bovine serum albumin; and
an instruction manual.

33. A method for detecting a breast cancer biomarker in a tissue sample with the SERS-nanotag according to claim 19, the method comprising:
(i) taking a paraffin embedded formalin fixed tissue sample;
(ii) washing the sample with xylene;
(iii) washing the sample of (ii) with absolute ethanol followed by washing with 95% ethanol followed by washing with 70% ethanol and then with 50% ethanol to obtain a washed tissue sample;
(iv) treating the washed tissue sample of (iii) with citrate buffer to obtain a treated tissue sample;
(v) incubating the treated tissue sample of (iv) with bovine serum albumin and washing with phosphate buffer saline;
(vi) incubating the tissue sample of (v) with the SERS-nanotag for 30 minutes and washing;
(vii) performing Raman spectroscopy on the tissue sample of (vi) to take signature peaks; and
(viii) analyzing the peaks to confirm presence of a breast cancer biomarker.

34. The method of claim 33, wherein the breast cancer biomarker is selected from the group consisting of Estrogen Receptor (ER), Progesterone Receptor (PR), Human Epidermal Growth Factor Receptor 2 (HER2), and Ki67.
